(19) United States
(12) Patent Application Publication
(10) Pub. No.: US 2010/0016293 A1
(43) Pub. Date: Jan. 21, 2010

(54) Quinazolines and Related Heterocyclic Compounds, and Their Therapeutic Use

(76) Inventors: Rogier Adriaan Smits, Amsterdam (NL); Herman Dirnawan Lim, Amsterdam (NL); Regorius Leurs, Amsterdam (NL); Iwan Jozef Philomena De Esch, Amsterdam (NL)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/307,197

(22) PCT Filed: Jul. 3, 2007
(86) PCT No.: PCT/EP07/56689

§ 371 Date: Sep. 9, 2009

(30) Foreign Application Priority Data
Jul. 3, 2006 (EP) 06076352.1

(51) Int. Cl. A61K 31/517; C07D 403/04; C07D 487/04; A61K 31/551

(52) U.S. Cl. 514/218; 544/291; 544/230; 540/575; 514/266.2; 514/252.17; 514/249; 514/266.22

(57) ABSTRACT

Compounds that interact with the histamine H4 receptor, and which may be useful for treating or preventing disorders and conditions mediated by the histamine H4 receptor, e.g. inflammation, are of formula (I) wherein Q is CR¹ or N; X is CR² or N, provided that Q and X are not both N; Y is CR³ or N; Z is CH or N; R¹, R², R³, R⁴, R⁵ and R⁶ are independently H, F, Cl, Br, I, or a hydrocarbon group which optionally contains one or more heteroatoms; and R7 is a heterocyclic radical including one or more N atoms; or a pharmaceutically acceptable salt, ester or solvate thereof.

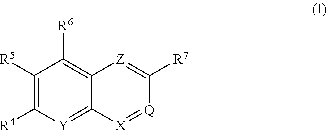

### FIELD OF THE INVENTION

The present invention relates to quinazolines and related heterocyclic compounds, and their therapeutic use. More particularly, it relates to compounds that interact with the histamine H₄ receptor, and to histamine H₄ receptor antagonists, and their use for treating, reducing or preventing disorders and discomforts mediated by the histamine H₄ receptor.

### BACKGROUND OF THE INVENTION

Histamine is important in human physiology because it is one of the chemicals released from certain cells (particularly mast cells) upon tissue injury or during the neutralisation of foreign material (e.g. antigens) by certain types of antibodies. Released histamine tends to dilate blood capillaries, often causing the skin to appear red and feel warm, and makes the capillaries more permeable, allowing fluid to escape into the surrounding tissue.

The biological activity of histamine is closely related with allergic responses and its deleterious effects, such as inflammation. Events that induce the inflammatory response include physical stimulation (including trauma), chemical stimulation, infection, and invasion by microorganisms. The inflammatory response is characterised by pain, increased temperature, redness, swelling, reduced function, itch, or any combination of these.

Mast cell degranulation (exocytosis) releases histamine and leads to an inflammatory response. A wide variety of immunological stimuli and non-immunological stimuli may cause the activation, recruitment and degranulation of mast cells. The activation of mast cells initiates allergic inflammatory responses, which in turn cause the recruitment of other effector cells that further contribute to the inflammatory response.

The numerous functions that are exerted by histamine are mediated through at least four pharmacologically distinct receptors, which are all members of the G-protein-coupled receptor family. The H₁ receptor is expressed in the brain, endothelial cells, and smooth muscle cells. Many of its functions contribute to allergic responses, and H₁ receptor antagonists have been very successful drugs for the treatment of allergies. The H₂ receptor has been demonstrated to function as a key modulator for gastric acid secretion, and H₂ receptor antagonists are widely used for the treatment of gastrointestinal ulcers. The H₃ receptor is predominantly expressed in the human central nervous system. It is believed to function as a presynaptic release-controlling receptor that may regulate histamine, norepinephrine, serotonin, GABA, acetylcholine, and other neurotransmitters. The histamine receptors couple to different signalling pathways via different G-proteins.

Recently, several groups have identified and characterised a fourth histamine receptor (see, e.g., T. Oda et al., J. Biol. Chem. 2000, 275(47), 36781-36786; C. Liu et al., Mol. Pharmacol. 2001, 59(3), 420-426; T. Nguyen et al., Mol. Pharmacol. 2001, 59(3), 427-433; Y. Zhu et al., Mol. Pharmacol. 2001, 59(3), 434-441; K. L. Morse et al., J. Pharmacol. Exp. Ther. 2001, 296(3), 1058-1066). The histamine H₄ receptor is a seven-transmembrane, G-protein-coupled receptor with approximately 40% homology to the histamine H₃ receptor. However, in contrast to the H₃ receptor, the H₄ receptor is expressed at greater levels in e.g. mast cells, eosinophils and a variety of other cells of the immune system.

It has been shown that administration of a histamine H₄ receptor antagonist inhibits histamine H₄ receptor-mediated calcium influx and chemotaxis of mast cells (Thurmond et al., J. Pharmacol. Exp. Ther. 2004, 309(1), 404-413) and eosinophils (Raible et al., Am. J. Respir. Crit. Care Med. 1994, 149(6), 1506-1511). This suggests an important role for the histamine H₄ receptor for the treatment of inflammatory diseases such as asthma, inflammatory bowel disease and several dermatological disorders. Further, histamine H₄ is associated with cancer and itch. See J. K. Bell et al., Br. J. Pharmacol. 2004, 142(2), 374-380; and F. Cianchi et al., Clin. Cancer Res. 2005, 11, 6807-6815.

US2005/0070527 describes 1H-quinoxaline compounds that inhibit leukocyte recruitment and modulate the H₄ receptor, and their use in treating conditions such as inflammation.

Further prior art in this general area includes WO2006/050965, WO02/072548, US20050070550A1, WO2007/031529, WO2004/022537A2, EP1767537A1 and US2006/0111416.

### SUMMARY OF THE INVENTION

Compounds according to the present invention, are of formula I

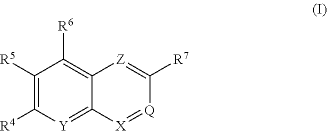

wherein

Q is CR¹ or N;

X is CR² or N, provided that Q and X are not both N;

Y is CR³ or N;

Z is CH or N;

R¹, R², R³, R⁴, R⁵ and R⁶ are independently H, F, Cl, Br, I, or a hydrocarbon radical which optionally contains one or more heteroatoms; and

R⁷ is a heterocyclic group including one or more N atoms or a pharmaceutically acceptable salt, ester or solvate thereof.

According to a further aspect of the invention, compounds of formula (I) can be used to treat, reduce or prevent disorders and discomforts mediated by the histamine H₄ receptor. For this purpose, they may be administered to a subject, e.g. as a pharmaceutical composition, in a therapeutically effective amount.

### DESCRIPTION OF THE INVENTION

Compounds of the invention may be chiral. This invention includes such compounds in any enantiomeric or diastereomeric form, including racemates. Compounds of the invention may also exist in different tautomeric forms, and all are included.

Preferably, in compounds of formula (I), R¹ and R² are independently selected from H, F, Cl, Br, I, C₁₋₄ alkyl, C₂₋₅ alkenyl, C₁₋₄ alkoxy, cycloalkyl, aryl (such as phenyl), heteroaryl, —C₁₋₄ alkyl-aryl such as benzyl or phenethyl, —C₁₋₄ alkyl-heteroaryl, such as heteroarylethyl, O-aryl such as O-phenylaryl, O-heteroaryl, NH-aryl such as NH-phenyl, NH-heteroaryl, S-aryl (such as S-phenyl), S-heteroaryl, O—C₁₋₄ alkyl-aryl such as O—CH₂-phenyl, O—(CH₂)₂-phenyl or O—(CH₂)₄-phenyl, O—C₁₋₄ alkyl-heteroaryl such as O—(CH₂)₂-heteroaryl or O—(CH₂)₄-heteroaryl, C₁₋₄ alkyl-heteroaryl such as CH₂CH₂-heteroaryl, NH—C₁₋₄ alkyl-aryl such as NHCH₂-phenyl, NH—C₁₋₄ alkyl-heteroaryl, hydrazino, hydroxylamino, NH₂, O—(CH₂)₃—N(CH₃)₂ and NR^{a}R^{b}, wherein either R^{a} is absent and R^{b} is acyl, or each of R^{a} and R^{b} is independently selected from H, C₁₋₄ alkyl, cycloalkyl, phenyl, benzyl and phenethyl, wherein any of said aryl, heteroaryl, alkyl, acyl, phenyl and cycloalkyl moieties is optionally substituted with 1 to 3 substituents, e.g. selected from C₁₋₄ alkyl, halogen, hydroxyl, amino and C₁₋₃ alkoxy.

Preferably, R³, R⁴, R⁵ and R⁶ are independently selected from H, F, Cl, Br, I, C₁₋₄ alkyl, C₂₋₅ alkenyl, C₂₋₅ alkynyl, C₁₋₄ alkoxy, C₁₋₄ alkylthio, C₃₋₆ cycloalkyl, O—C₃₋₆ cycloalkyl, phenyl, benzyl, O-phenyl, NH-phenyl, S-phenyl, O—C₁₋₄ alkyl-phenyl such as O—(CH₂)₂-phenyl or O—(CH₂)₄-phenyl, C₁₋₄ alkyl-aryl such as CH₂CH₂-phenyl, CF₃, O—CF₃, S—CF₃, hydroxy, nitro, cyano, O—C₁₋₄ alkyl-N(CH₃)₂, O—(CH₂)₃—N(CH₃)₂ and NR^{a}R^{b}, wherein each of R^{a} and R^{b} is independently selected from H, C₁₋₄ alkyl, phenyl, benzyl and phenethyl, and wherein any phenyl, alkyl or cycloalkyl moiety is optionally substituted with 1 to 3 substituents selected from C₁₋₃ alkyl, halogen, hydroxy, amino and C₁₋₃ alkoxy.

As indicated above, R⁷ is a heterocyclic radical comprising one or more N atoms. It is bonded to the bicyclic nucleus via a N or C atom, bonding via N being preferred. This radical may be mono- or bi-cyclic, and optionally carries substituents, e.g. substituents as defined above.

By way of example, R⁷ is selected from 4-7 membered heterocyclyl, C₃₋₇ cycloalkyl-4-7 membered heterocyclyl and bis-(4-7 membered heterocyclyl). In a preferred embodiment, R⁷ is selected from cyclic amines, spiroamines and bridged cycloamines.

R⁷ may in particular be selected from any of the following groups

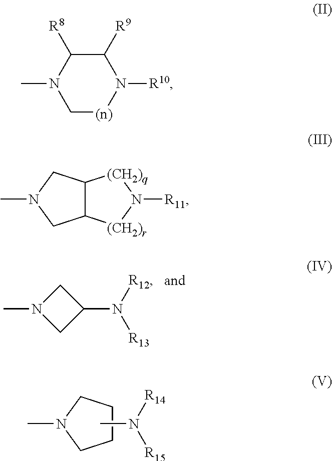

wherein

n is 1 or 2;

R⁸ and R⁹ are independently H or C₁₋₃ alkyl;

R¹⁰ is H, C₃₋₅ alkenyl with no sp²-carbon member attached directly to the R¹⁰-attached nitrogen member, C₃₋₅ alkynyl with no sp-carbon member attached directly to the R¹⁰-attached nitrogen member, CH₂CH₂OH, C₁₋₄ alkyl-O—C₁₋₄ alkyl or C₁₋₆ alkyl which is optionally substituted by halogen, cyano, hydroxy, carboxy, amino, C₁₋₆ alkylamino, N,N-di(C₁₋₆ alkyl)amino, C₁₋₆ alkylthio, C₁₋₆ alkoxy or C₃₋₈ cycloalkyl;

alternatively R¹⁰ may be taken together with R⁹, wherein the R⁹-attached carbon member, and the R¹⁰-attached nitrogen member form a 5-, 6-, or 7-membered heterocyclic ring, wherein said ring has 0 or 1 additional heteroatoms selected from O, S, NH and NC₁₋₆ alkyl, and wherein said heterocyclic ring is substituted with 0, 1, 2 or 3 substituents each selected from C₁₋₃ alkyl, halogen, hydroxy, amino and C₁₋₃ alkoxy;

q is 1, 2 or 3;

r is 0 or 1;

R¹¹ is a hydrogen atom or C₁₋₆ alkyl optionally substituted by halogen, cyano, hydroxy, carboxy, amino, N—(C₁₋₆ alkyl)amino, N,N-di(C₁₋₆ alkyl)amino, C₁₋₆ alkylthio, C₁₋₆ alkoxy, or C₃₋₈ cycloalkyl; and

R¹², R¹³, R¹⁴ and R¹⁵ are independently hydrogen or C₁₋₆ alkyl optionally substituted by halogen, cyano, hydroxy, carboxy, amino, C₁₋₆ alkylamino, N—(C₁₋₆ alkyl)amino, N,N-di(C₁₋₆ alkyl)amino, C₁₋₆ alkylthio, C₁₋₆ alkoxy, or C₃₋₈ cycloalkyl.

The following aminergic substituents are examples of R⁷:

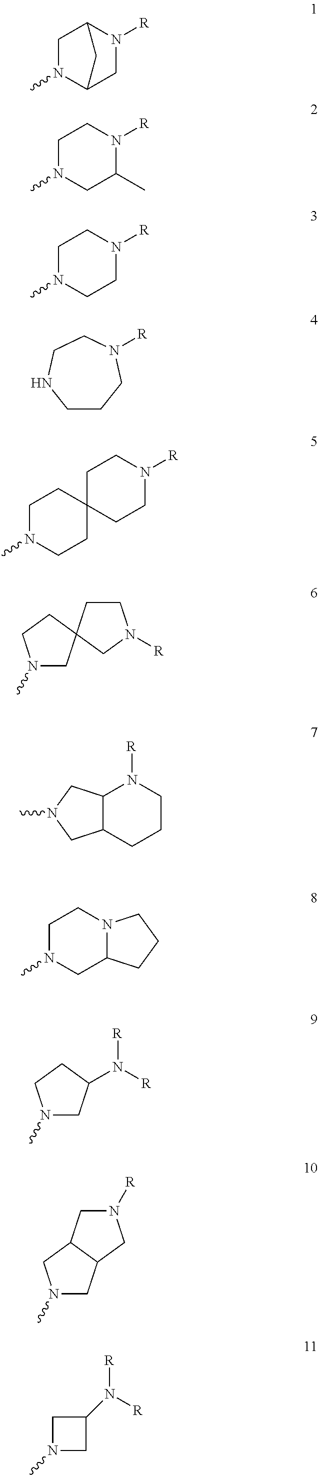

wherein R is H or any substituent, e.g. methyl or ethyl, and the curved lines represent the bond of the amine group with the heterocyclic scaffold. Amines 5 and 6 are examples of spiroamines. Amines 7, 8, 9 and 10 are examples of compounds that have stereoisomers, of which all forms (e.g. S- and R-isomers) are included.

As indicated above, R⁷ may also be linked to the bicyclic nucleus via a C atom. Examples of such groups are 7-octahydroindolizinyl and 1-methyl-4-piperidinyl.

The broad nature of the group R⁷ is illustrated in the prior art documents identified above. The content of each is incorporated herein by reference.

The term “alkyl” as used herein includes straight-chain and branched hydrocarbon groups.

The term “alkenyl” as used herein includes straight-chain and branched hydrocarbon groups as above with at least one carbon-carbon double bond (sp²).

The term “alkynyl” as used herein includes straight-chain and branched hydrocarbon groups as above with at least one carbon-carbon triple bond (sp). Hydrocarbons having a mixture of double bonds and triple bonds are grouped as alkynyls herein.

The term “alkoxy” as used herein includes straight-chain and branched alkyl groups with a terminal oxygen linking the alkyl group to the rest of the molecule.

The term “aryl” as used herein includes any functional group or substituent comprising an aromatic ring. In particular the aryl may be selected from moieties comprising a phenyl, naphtyl or biphenyl. The aryl may comprise one or more heteroatoms, in which case the aryl may be referred to as “heteroaryl”. Preferred examples of heteroaryl groups include pyridine, furane, thiophene, triazole and tetrazole.

Any hydrocarbon radical may be, for example, a C₁₋₂₀ hydrocarbon, preferably a C₁₋₁₂ hydrocarbon, and more preferably a C₁₋₁₀ hydrocarbon. This range applies also to other groups, including heterocyclic groups, and also as a preference.

It is understood that substitutions and combinations of substitutions recited herein refer to substitutions that are consistent with the valency of the member being substituted.

The “pharmaceutically acceptable salt, ester or solvate thereof” refers to those salts, ester forms and solvates of the compounds of the present invention that would be apparent to the pharmaceutical chemist, i.e. those that are non-toxic and that would favourably affect the pharmacological properties of said compounds of the present invention. Those compounds having favourable pharmacological properties would be apparent to the pharmaceutical chemist, i.e. those that are non-toxic and that possess such pharmacological properties to provide sufficient palatability, absorption, distribution, metabolism and excretion. Other factors, more practical in nature, that are important in the selection are cost of raw materials, ease of crystallisation, yield, stability, hygroscopicity, and flowability of the resulting bulk drug.

Representative acids that may be used in the preparation of pharmaceutically acceptable salts include but are not limited to the following: acetic acid, 2,2-dichlorolactic acid, acylated amino acids, adipic acid, alginic acid, ascorbic acid, L-aspartic acid, benzenesulphonic acid, benzoic acid, 4-acetamidobenzoic acid, (+)-camphoric acid, camphorsulphonic acid, (+)-(1S)-camphor-10-sulphonic acid, capric acid, caproic acid, caprylic acid, cinnamic acid, citric acid, cyclamic acid, dodecylsulphuric acid, ethane-1,2-disulphonic acid, ethanesulphonic acid, 2-hydroxy-ethanesulphonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hipuric acid, hydrobromic acid, hydrochloric acid, (+)-L-lactid acid, (±)-DL-lactic acid, lactobionic acid, maleic acid, (−)-L-malic acid, malonic acid, (±)-DL-mandelic acid, methanesulphonic acid, naphthalene-2-sulphonic acid naphthalene-1,5-disulphonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulphuric acid, tannic acid, (+)-L-tartaric acid, thiocyanic acid, p-toluenesulphonic acid and undecylenic acid.

Representative bases that may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Examples of suitable esters include C₁₋₇ alkyl, C₅₋₇ cycloalkyl, phenyl, substituted phenyl, and phenyl-C₁₋₆ alkyl esters. Preferred esters include methyl esters.

If R⁷ is the group of formula (II), n is preferably 1. In a preferred embodiment, R⁸ and R⁹ are hydrogen atoms, while R¹⁰ is a methyl group.

If R⁷ is the group of formula (III), preferred combinations of q and r are for example q=1 and r=1, q=2 and r=0, and q=3 and r=0.

A preferred class of compounds according to the invention is quinoxaline compounds of general formula (I), wherein Q is CR¹, X is N, Y is CR³, and Z is N. Particularly preferred compounds are those quinoxalines wherein at least three groups out of R³, R⁴, R⁵ and R⁶ are hydrogen atoms. Further preferred compounds are those quinoxalines wherein R⁷ is 4-methylpiperazino.

Another preferred class of compounds is quinazoline compounds of general formula (I), wherein Q is N, X is CR², Y is CR³, and Z is N. Particularly preferred compounds are those quinazolines wherein at least three groups out of R³, R⁴, R⁵ and R⁶ are hydrogen atoms. Further preferred compounds are those quinazolines wherein R⁷ is 4-methylpiperazino.

A further preferred class of compounds is quinoline compounds of general formula (I), wherein Q is CR¹, X is CR², Y is CR³, and Z is N. Particularly preferred compounds are those quinolines wherein R¹, R², R³, R⁵ and R⁶ are hydrogen atoms. It is further preferred that R⁷ is 4-methylpiperazino.

Yet another preferred class of compounds is isoquinoline compounds of general formula (I), wherein Q is N, X is CR², Y is CR³, and Z is CH. Particularly preferred compounds are isoquinolines wherein R¹, R², R³, R⁴, R⁵ and R⁶ are hydrogen atoms. Further preferred compounds are those isoquinolines wherein R⁷ is 4-methylpiperazino.

Tables 1-5 illustrate compounds of the invention.

TABLE 1

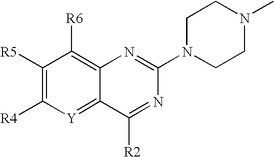

| Compound | Y | R² | R⁴ | R⁵ | R⁶ |
|---|---|---|---|---|---|
| VUF10249 | CH | H | H | H | H |
| VUF10328 | CH | OCH₂-phenyl | H | H | H |
| VUF10327 | CH | O-phenyl | H | H | H |
| VUF10331 | CH | OH | Cl | H | H |
| VUF10147 | CH | NH₂ | H | H | H |
| VUF10247 | CH | NH-phenyl | H | H | H |
| VUF10246 | CH | NHCH₂-phenyl | H | H | H |
| VUF10332 | CH | NH(CH₂)₂-phenyl | H | H | H |
| VUF10329 | CH | NHCH₂-phenyl | Cl | H | H |
| VUF10349 | CH | NHCH₃ | Cl | H | H |
| VUF10350 | CH | N(CH₃)₂ | Cl | H | H |
| VUF10351 | CH | NH₂ | Cl | H | H |
| VUF10453 | CH | NH(CH₂)₂-phenyl | Cl | H | H |
| VUF10452 | CH | NHCH(CH₃)₂ | Cl | H | H |
| VUF10455 | CH | NH(CH₂)₂—OH | Cl | H | H |
| VUF10456 | CH | NHCH₂COOC₂H₅ | Cl | H | H |
| VUF10506 | CH | NH(CH₂)₂-(4- | Cl | H | H |
| | | nitrophenyl) | | | |
| VUF10509 | CH | NHCH₂-(4- | Cl | H | H |
| | | aminophenyl) | | | |
| VUF10505 | CH | NHCH₂-(4- | Cl | H | H |
| | | methoxyphenyl) | | | |
| VUF10513 | CH | NHCH₂-(4- | Cl | H | H |
| | | fluorophenyl) | | | |
| VUF10553 | CH | NHCH₂-(4- | Cl | H | H |
| | | cyanophenyl) | | | |
| VUF10556 | CH | NHCH₂-(3,4- | Cl | H | H |
| | | difluorophenyl) | | | |

TABLE 2

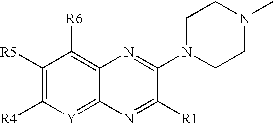

| Compound | R¹ | R³ | R⁴ | R⁵ | R⁶ |
|---|---|---|---|---|---|
| VUF10148 | CH₂-phenyl | H | H | H | H |
| VUF10171 | CH₂-phenyl | CH₃ | H | H | H |
| VUF10178 | CH₂-phenyl | H | H | H | CH₃ |
| VUF10210 | CH₂-phenyl | H | CH₃ | CH₃ | H |
| VUF10241 | CH₂CH₂-phenyl | H | Cl | Cl | H |
| VUF10240 | CH₂CH₂-phenyl | H | CH₃ | CH₃ | H |
| VUF10170 | O-phenyl | H | H | H | H |
| VUF10199 | S-phenyl | H | H | H | H |
| VUF10277 | O-phenyl | H | Cl | Cl | H |
| VUF10050 | H | H | H | H | H |
| VUF6886 | H | H | Cl | H | H |
| VUF10166 | Cl | H | H | H | H |
| VUF10144 | CH₃ | H | H | H | H |
| VUF10146 | Phenyl | H | H | H | H |
| VUF10059 | OCH₂CH₃ | H | H | H | H |
| VUF10180 | OCH₂-phenyl | H | H | H | H |
| VUF10182 | O(CH₂)₂-phenyl | H | H | H | H |
| VUF10215 | O(CH₂)₄-phenyl | H | H | H | H |
| VUF10216 | OCH₂CH(CH₃)₂ | H | H | H | H |
| VUF10181 | O-cyclohexyl | H | H | H | H |
| VUF10212 | O-4-chlorophenyl | H | H | H | H |
| VUF10211 | O-4-fluorophenyl | H | H | H | H |
| VUF10324 | O-4-methylphenyl | H | H | H | H |
| VUF10318 | O-4-methoxyphenyl | H | H | H | H |
| VUF10213 | O-3-methylphenyl | H | H | H | H |
| VUF10322 | O-3-N,N- | H | H | H | H |
| | dimethylaminophenyl | | | | |
| VUF10200 | O(CH₂)₃N(CH₃)₂ | H | H | H | H |
| VUF10198 | O-3,4-dichlorophenyl | H | H | H | H |
| VUF10274 | O(CH₂)₂-2-pyridyl | H | H | H | H |
| VUF10275 | O(CH₂)₂-3-pyridyl | H | H | H | H |
| VUF10276 | O(CH₂)₂-4-pyridyl | H | H | H | H |
| VUF10279 | O-(3-pyridyl) | H | H | H | H |
| VUF10352 | Methoxy | H | Cl | Cl | H |
| VUF10278 | NHCH₂-phenyl | H | H | H | H |
| VUF10319 | OCH₂-(4-methoxyphenyl) | H | H | H | H |
| VUF10323 | OCH₂-(4-chlorophenyl) | H | H | H | H |
| VUF10334 | OCH₂-(3-chlorophenyl) | H | H | H | H |
| VUF10325 | OCH₂-(4-methylphenyl) | H | H | H | H |
| VUF10330 | Methoxy | H | H | H | H |
| VUF10321 | Methoxy | H | Cl | H | H |
| VUF10348 | Methyl | H | Cl | Cl | H |
| VUF10353 | Trifluoromethyl | H | H | H | H |

TABLE 3

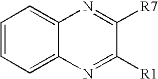

| Compound | R¹ | R⁷ |
|---|---|---|
| VUF10111 | Cl | 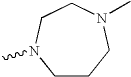 |
| VUF10145 | Cl | 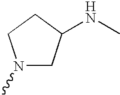 |
| VUF10217 | CH₂-phenyl | 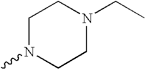 |
| VUF10365 | H | 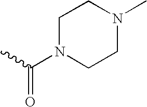 |
| VUF10366 | H | 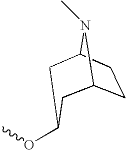 |

TABLE 4

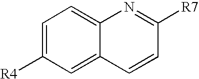

| Compound | R⁴ | R⁷ |
|---|---|---|
| VUF10049 | H | 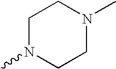 |
| VUF6959 | Cl | 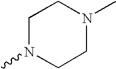 |
| VUF6957 | H | 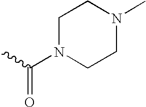 |

TABLE 5

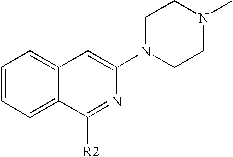

| Compound | R² |
|---|---|
| VUF10047 | H |
| VUF10364 | NH₂ |

The present invention includes prodrugs of the compounds of the invention. In general, such prodrugs will be functional derivatives of the compounds that are readily convertible in vivo into the bio-active compound. Thus, in the uses of the compounds for methods of treatment of the present invention, the term “administering” shall encompass the treatment of the various disorders described with the compound specifically disclosed or with a compound that may not be specifically disclosed, but that converts to the specified compound in vivo after administration to the patient. Analogously, the term “compound”, when applied to compounds of this invention, shall encompass any specific compound according to the present invention or any compound (or prodrug) that converts to the specifically disclosed compound in vivo after administration, even if such prodrug is not explicitly disclosed herein.

Compounds of the present invention are antagonists, inverse agonists or partial agonists of histamine H₄ receptors. Thus, these compounds reversibly or irreversibly bind to the histamine H₄ receptor. Without wishing to be bound by theory, this is considered to be indicative of therapeutic utility.

The effect of an antagonist may also be obtained by an inverse agonist or a partial agonist. Inverse agonism describes the property of a compound to actively turn off a receptor that displays constitutive activity. Constitutive activity can be identified in cells that have been forced to over-express the human H₄ receptor. Constitutive activity can be measured by measuring cAMP (cyclic adenosine monophosphate) levels or by measuring a reporter gene sensitive to cAMP levels after a treatment with a cAMP stimulating agent such as forskolin. Cells that over-express H₄ receptors will display lower cAMP levels after forskolin treatment than non-expressing cells. Compounds that behave as H₄ agonists will dose-dependently lower forskolin-stimulated cAMP levels in H₄-expressing cells. Compounds that behave as H₄ inverse agonists will dose-dependently stimulate cAMP levels in H₄-expressing cells. Compounds that behave as H₄ antagonists will block either H₄ agonist induced inhibition of cAMP or H₄ inverse agonist induced increases in cAMP.

Compounds of the invention may be administered to a subject, in therapy, e.g. for the treatment of inflammation.

“Inflammation” herein refers to the response that develops as a consequence of release of inflammatory mediators, such as histamine, serotonin, leukotrienes, prostaglandins, cytokines, chemokines, which in turn is caused by at least one stimulus, which can be for example an immunological stimulus or a non-immunological stimulus.

The term “subject” as used in this application includes animals and in particular mammals including and preferably being a human, a dog, a cat, a horse, a rat, a rabbit, a mouse, and a non-human primate, which animal is in need of observation, experiment, treatment or prevention in connection with the relevant disease or condition.

The term “composition” as used in this application includes a product comprising the specified ingredients in the specified amounts, including in the therapeutically effective amounts, as well as any product that results directly or indirectly from combinations of the specified ingredients in the specified amounts.

The term “therapeutically effective amount” as used in this description and the appending claims is meant to be that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The compounds of the invention are useful for the amelioration of symptoms associated with the treatment and/or the prevention of conditions and diseases such as inflammatory disorders, allergic disorders, dermatological disorders, autoimmune disease, lymphatic disorders, immunodeficiency disorders and cancer, including the more specific conditions and diseases given above.

The invention is also directed to a pharmaceutical composition for treating or preventing an H₄ receptor-mediated condition in a subject, comprising a therapeutically effective amount for treating, reducing or preventing an H₄ receptor-mediated condition of at least one H₄ receptor antagonist or partial agonist or inverse agonist according to the present invention. Such pharmaceutical compositions typically also comprise a pharmaceutically acceptable carrier.

In addition, the invention features an anti-inflammatory composition, comprising a therapeutically effective amount for treating or preventing inflammation of at least one anti-inflammatory compound according to the present invention. These compositions typically also comprise a pharmaceutically acceptable carrier.

Another example of the invention is the use of a compound according to the present invention in the preparation of a medicament for treating any one of the conditions referred to herein; one of such conditions is inflammation. Another example of the invention is the use of a compound according to the present invention in the treatment or prevention of any one of the conditions referred to herein; one of such conditions is inflammation.

The invention is also directed to a method for treating or preventing inflammation in a subject, comprising administering to a subject in connection with an inflammatory response a pharmaceutical composition that comprises a therapeutically effective amount of at least one anti-inflammatory compound according to the present invention.

The invention also features methods for treating or preventing an H₄ receptor-mediated condition in a subject, comprising administering to the subject a pharmaceutical composition that comprises a therapeutically effective amount of at least one H₄ receptor antagonist, partial agonist or inverse agonist according to the present invention.

Embodiments of methods for treating or preventing inflammation in a subject that comprise administering to the subject in connection with an inflammatory response a pharmaceutical composition comprising a therapeutically effective amount of at least one anti-inflammatory compound according to the present invention include methods wherein at least one of the following is satisfied: said inflammatory response is a response to a physical stimulus; said inflammatory response is a response to a chemical stimulus; said inflammatory response is a response to infection; said inflammatory response is a response to an invasion by a body that is foreign to said subject; said inflammatory response is a response to an immunological stimulus; said inflammatory response is a response to a non-immunological stimulus; said inflammatory response is a response to at least one of the conditions: allergy, asthma, chronic obstructed pulmonary disease, atherosclerosis, rheumatoid arthritis, multiple sclerosis, inflammatory bowel disease, and more specifically wherein said inflammatory bowel disease is at least one of Crohn's disease and ulcerative colitis, psoriasis, allergic rhinitis, scleroderma, autoimmune thyroid disease, immune-mediated diabetes mellitus, and lupus; said inflammatory response is a response to at least one of the conditions: myasthenia gravis, autoimmune neuropathy, and more specifically wherein said autoimmune neuropathy is Guillain-Barré neuropathy, autoimmune uveitis, autoimmune haemolytic anemia, pernicious anemia, autoimmune thrombocytopenia, temporal arteritis, anti-phospholipid syndrome, vasculitides, and more specifically wherein said vasculitides is Wegener's granulomatosis, Behcet's disease, dermatitis herpetiformis, pemphigus vulgaris, vitiligio, primary biliary cirrhosis, autoimmune hepatitis, autoimmune oophoritis, autoimmune orchitis, autoimmune disease of the adrenal gland, polymyositis, dermatomyositis, spondyloarthropathy, and more specifically wherein said spondyloarthropathy is ankylosing spondylitis, and Sjogren's syndrome; said inflammatory response is acute inflammation; said inflammatory response is chronic inflammation. Moreover, the compounds of the invention can be used in the treatment of or therapy against cancer. In yet a further embodiment the compounds of the invention can be used to reduce, suppress or avoid itch. Administration “in connection with” an inflammatory response according to the present invention includes administration at a time that it is at least one of prior to, at the onset of, and after inflammation is detected.

Aspects of the invention include (a) a pharmaceutical composition comprising at least one compound according to the invention and a pharmaceutically acceptable carrier; (b) a packaged drug comprising (1) a pharmaceutical composition comprising at least a compound according to the present invention and a pharmaceutically acceptable carrier, and (2) instructions for the administration of said composition for the treatment or prevention of any one of the conditions referred to herein, such as an H₄-mediated disease or condition, and more particularly inflammation.

This invention provides methods for treating, reducing or preventing an H₄-mediated condition in a subject, said methods comprising administering to the subject a pharmaceutically effective amount of a composition comprising at least one compound according to the invention. In these conditions, the action of the H₄ receptor is involved. For example, the invention features a method for treating an H₄ mediated condition in a subject, said method comprising administering to the subject a pharmaceutically effective H₄-antagonising amount of a composition comprising at least one compound according to the invention. As used herein “treating” a disorder means eliminating, reducing or otherwise ameliorating the cause and/or effects thereof. Terms such as to “inhibit” the onset of a disorder or event, and to “prevent” a disorder or condition mean preventing, delaying or reducing the likelihood of such onset.

The term “unit dose” is used herein to refer to physically discrete units suitable as unitary dosages for subjects, each unit containing a predetermined effective, pharmacologic effective amount of the active ingredient calculated to produce the desired pharmacological effect. The specifications for the novel unit dosage forms of this invention are determined by, and are directly dependent on, the characteristics of the active ingredient, and on the limitations inherent in the art of compounding such an active ingredient for therapeutic use in subjects.

The pharmaceutical compositions can be prepared using conventional pharmaceutical excipients and compounding techniques. Examples of suitable unit dosage forms are tables, capsules, pills, powders, powder packets, granules, wafers, and the like, segregated multiples of any unit dosage form, as well as liquid solutions, and suspensions. Some liquid forms are aqueous, whereas other embodiments of liquid forms are non-aqueous. Oral dosage forms may be elixirs, syrups, capsules, tablets and the like. Examples of solid carriers include those materials usually employed in the manufacture of pills or tablets, such as lactose, starch, glucose, methylcellulose, magnesium stearate, dicalcium phosphate, mannitol and the like, thickeners such as tragacanth and methylcellulose USP, finely divided SiO₂, polyvinylpyrrolidone, magnesium stearate, and the like. Typical liquid oral excipients include ethanol, glycerol, water and the like. All excipients may be mixed as needed with diluents (for example, sodium and calcium carbonates, sodium and calcium phosphates, and lactose), disintegrants (for example, cornstarch and alginic acid), granulating agents, lubricants (for example magnesium stearate, stearic acid, and talc), binders (for example starch and gelatine), thickeners (for example paraffin, waxes, and petrolatum), flavouring agents, colouring agents, preservatives, and the like by conventional techniques known to those of ordinary skill in the art of preparing dosage forms. Coatings can be present and include for example glyceryl monostearate and/or glyceryl distearate. Capsules for oral use include hard gelatine capsules in which the active ingredient is mixed with a solid diluent, and soft gelatine capsules, in which the active ingredient is mixed, with water or an oil, such as peanut oil, liquid paraffin, or olive oil.

Parenteral dosage forms may be prepared using water or another sterile carrier. Parenteral solutions can be packaged in containers adapted for subdivision into individual doses. For intramuscular, intraperitoneal, subcutaneous, and intravenous use, the compounds according to the invention will be generally provided in sterile aqueous solutions or suspensions, buffered to an appropriate pH and isotonicity. Suitable aqueous vehicles include Ringer's solution and isotonic sodium chloride. Aqueous suspensions may include suspending agents such as cellulose derivatives, sodium alginate, polyvinyl-pyrrolidone, and gum tragacanth, and a wetting agent, such as lecithin. Suitable preservatives for aqueous suspensions include ethyl and n-propyl p-hydroxybenzoate. Parenteral formulations include pharmaceutically acceptable aqueous or non-aqueous solutions, dispersions, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Carriers for solid dosage forms include (a) fillers or extenders, (b) binders, (c) humectants, (d) disintegrating agents, (e) solution retarders, (f) absorption accelerators, (g) adsorbants, (h) lubricants, (i) buffering agents, and (j) propellants.

Compositions may also contain adjuvants such as preserving, wetting, emulsifying, and dispensing agents; antimicrobial agents such as parabens, chlorobutanol, phenol, and sorbic acid; isotonic agents such as sugar or sodium chloride; absorption-prolonging agents such as aluminium monostearate and gelatine; and absorption-enhancing agents.

Physiologically acceptable carriers are well known in the art. Examples of liquid carriers are solutions in which compounds according to the invention form solutions, emulsions, and dispersions. Compatible antioxidants, such as methylparaben and propylparaben, can be present in solid and/or liquid compositions, as can sweeteners.

Pharmaceutical compositions according to the invention may include suitable emulsifiers typically used in emulsion compositions. Gelling agents may also be added to compositions according to this invention. Polyacrylic acid derivatives, such as carbomers, are examples of gelling agents, and more particularly, various types of carbopol. Suspensions may be prepared as a cream, an ointment, including a water-free ointment, a water-in-oil emulsion, an oil-in-water emulsion, and emulsion gel, or a gel.

Compounds according to the present invention can be administered by oral or parenteral routes, including intravenous, intramuscular, intraperitoneal, subcutaneous, rectal, intracisternal, intravaginal, intravesical, topical or local administration, and by inhalation (bucal or nasal, preferably in the form of a spray). For oral administration, the compounds according to the invention will be generally provided in the form of tablets, capsules, or as a solution or suspension. Other methods of administration include controlled release formulations, such as subcutaneous implants and dermal patches.

Pharmaceutically effective doses of the compounds according to the invention may be ascertained by conventional methods. The specific dosage level required for any particular subject will depend on a number of factors, including severity of the condition, type of symptoms needing treatment, the route of administration, the weight, age, and general condition of the subject, and the administration of other medicaments.

In general, the daily dose (whether administered as a single dose or as divided doses) will be in the range of from about 0.01 mg to about 1000 mg per day, more usually from about 1 mg to about 500 mg per day, and most usually from about 10 mg to about 200 mg per day. Expressed as dosage per unit body weight, a typical dose will be expected to be between about 0.0001 mg/kg and about 15 mg/kg, especially between about 0.01 mg/kg and about 7 mg/kg, and most especially between about 0.15 mg/kg and 2.5 mg/kg.

Oral dose ranges include from about 0.01 to 500 mg/kg, daily, more preferably from about 0.05 to about 100 mg/kg, taken in 1-4 separate doses. Some compounds of the invention may be orally dosed in the range of about 0.05 to about 50 mg/kg daily, while others may be dosed at 0.05 to about 20 mg/kg daily. Infusion doses can range from about 1.0 to about 1.0×10⁴ μg/(kg·min) of inhibitor, admixed with a pharmaceutical carrier over a period ranging from several minutes to several days. For topical administration, compounds of the present invention may be mixed with a pharmaceutical carrier at a concentration from about 0.1 to about 10% of drug to vehicle. Capsules, tablets or other formulations (such as liquids and film-coated tablets) may be of between 0.5 and 200 mg, such as 1, 3, 5, 10, 15, 25, 35, 50 mg, 60 mg, and 100 mg and can be administered according to the disclosed methods. Daily dosages are envisaged to be, for example between 10 mg and 5000 mg for an adult human subject of normal weight.

The compounds of the invention can be prepared according to processes within the skill of the art and/or according to processes of this invention, such as those described in the schemes and examples that follow and by matrix or combinatorial methods.

Illustrative preparations are presented in the following Schemes. Other compounds of the invention may be made in the same general way, using modifications that will be apparent to those of ordinary skill in the art.

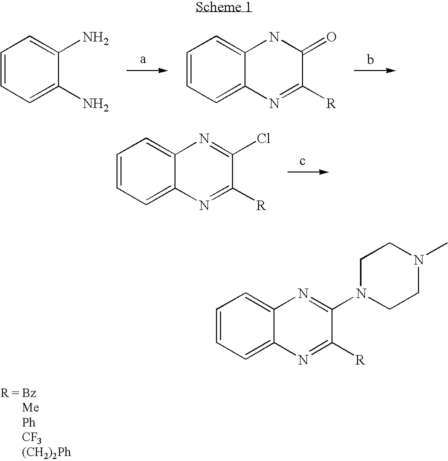

a) phenylpyruvic acid, ETOH, reflux; b) POCl₃, reflux; c) N-methylpiperazine, mw, 140° C. The compounds were prepared starting from o-phenylenediamine with phenylpyruvic acid, pyruvic acid, glyoxylic acid, trifluoropyruvic acid or oxobutyric acid ethyl ester (reaction carried out at room temperature) respectively.

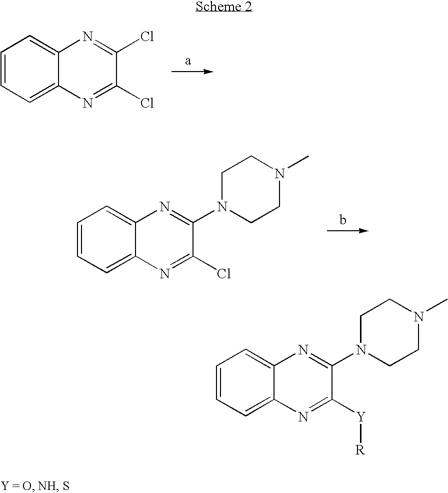

### a) N-methylpiperazine, DIPEA, THF, mw, 160° C.; b) NaH, RYH, DMF, r.t.

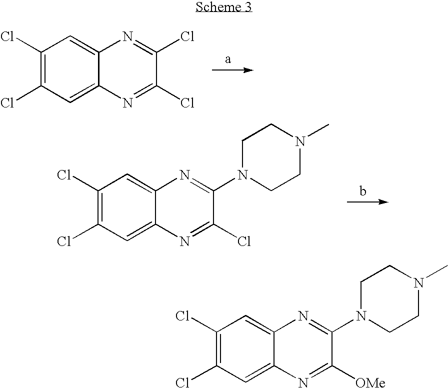

a) N-methylpiperazine, DIPEA, THF, mw, 160° C.; b) NaOMe, MeOH, reflux.

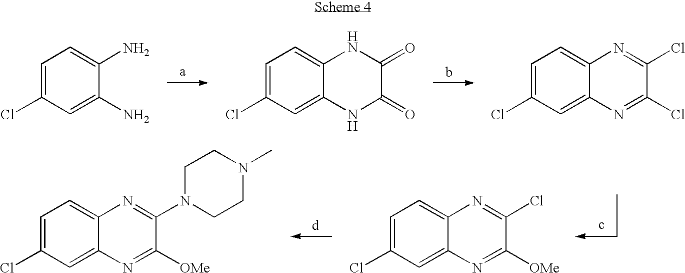

a) diethyl oxalate, reflux; b) POCl₃, reflux; c) NaOMe, MeOH, 50° C.; d) N-methylpiperazine, DIPEA, THF, mw, 160° C.

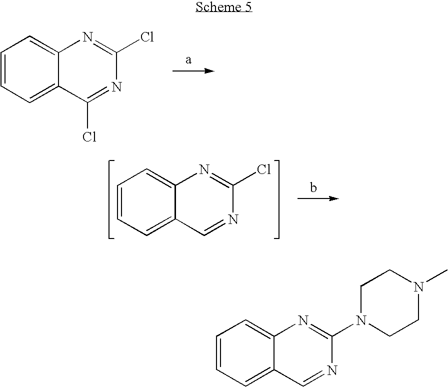

a) brine with 9% NH₃OH, Zn, DCM, reflux; b) N-methylpiperazine, mw, 140° C.

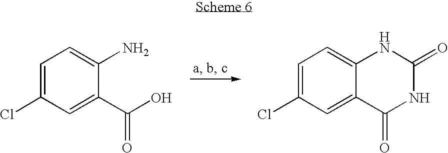

a) KNCO, water; b) NaOH, NaCl; c) H₂SO₄.

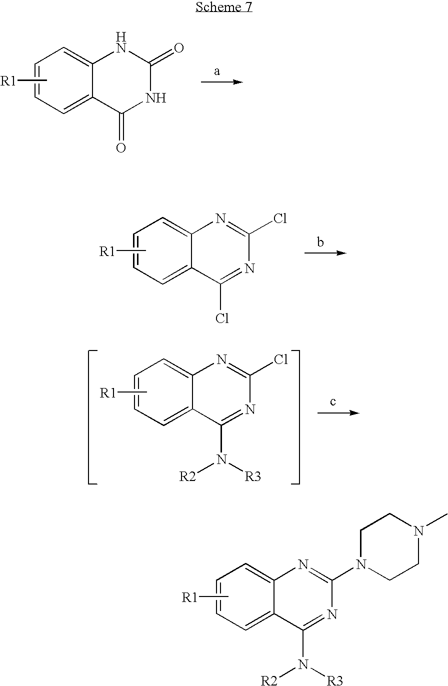

a) N,N-diethylaniline, POCl₃, reflux; b) NR₂R₃, various solvents, r.t., c) N-methylpiperazine, EtOAc, mw, 140° C.

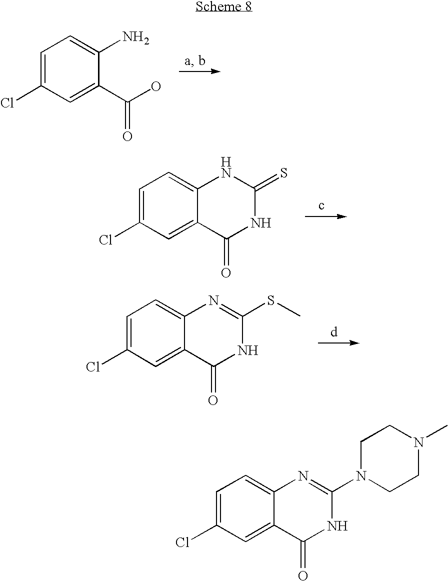

a) SOCl₂, reflux; b) NH₄SCN, acetone, r.t.; c) CH₃I, NaOH, water, r.t.; d) N-methylpiperazine, reflux.

The following Examples illustrate the invention, and also the preparation of intermediates.

Chemicals and reagents were obtained from commercial suppliers and were used without further purification. Yields given are isolated yields unless mentioned otherwise. Flash column chromatography was typically carried out on an Argonaut Flashmaster™ II flash chromatography system, using prepacked Isolute Flash Si II columns with the UV detector operating at 254 nm. All meltingpoints are uncorrected and were measured on an Optimelt Automated Melting Point System from Stanford research systems. All ¹H NMR and ¹³C NMR spectra were measured on a Brüker 200.

Analytical HPLC-MS analyses were conducted using a Shimadzu LC-8A preparative liquid chromatograph pump system with a Shimadzu SPD-10AV UV-VIS detector with the MS detection performed with a Shimadzu LCMS-2010 liquid chromatograph mass spectrometer. The analyses were performed using the following two conditions:

Condition I: an Xbridge™ (C18) 5μ column (100 mm×4.6 mm) with the following two solvents; solvent A, 90% MeCN-10%; solvent B, 90% water-10% buffer; flow rate=2.0 ml/min; Start 95% A, linear gradient to 90% B in 10 min, then 10 min at 90% B, then 10 min at 95% A. Total run time 30 min.

Analytical HPLC-MS analyses for condition II were carried out on Agilent 1100 Series HPLC-MS-System including an Agilent G1315B DAD. Condition II: XBridge™ (C18) 3.5 μm column (2.1 mm×50 mm) with the following two solvents: solvent A, a 5 mM solution of NH₄HCO₃ in water set to pH 9.0 using 19 mM NH₃; solvent B, 100% ACN. The MS detection was performed with an Agilent G1956B LC/MSD SL using a multimode ion source. Flow rate=1.2 ml/min; Start: 95% A, linear gradient to 5% A in 1.25 min, then 0.75 min at 5% A, followed by 1.0 min 95% A, 5% B. Total runtime: 3 min.

Preparative HPLC-MS separations were carried out on an Agilent 1100 Series preparative HPLC-MS System including a G1968D active splitter, G1315B DAD and a G1946D LC/MSD using an ESI ion source. Elution over a Waters X-Terra MS C18 5μ column (19 mm×100 mm) with the following two solvents: solvent A, a 10 mM solution of NH₄HCO₃ in water set to pH 9.5 using 38 mM NH₃; solvent B, 100% ACN; flow rate: 30 ml/min; exemplary gradient description (30% B->75% B): start: 70% A, 30% B, 1.3 min isocratic elution, 6.0 min to 25% A, 75% B followed by 0.7 min to 95% A, 5% B, then 1.0 min at 5% A and 95% B, followed by 1.8 min 75% A, 30% B. Total runtime: 10.8 min.

2,4,6-Trichloroquinazoline can be prepared according to a procedure described in the scientific literature (J. Med. Chem. 1995 p. 3547-3557). This procedure can also be used for the synthesis of 2,4,7-trichloroquinazoline, another precursor of compounds of the invention.

### Example 1

### 4-Amino-6-chloro-2-(4-methylpiperazinyl)quinazoline (VUF10247)

2,4,6-Trichloroquinazoline (300 mg) was added to a saturated solution of ammonia in MeOH (5.0 mL) and stirred at room temperature (r.t.). After 16 hours the mixture was diluted with EtOAc (50 mL) and washed with water and brine. After drying over Na₂SO₄ the organic phase was concentrated (about 3 mL) and transferred to a microwave tube containing N-methylpiperazine (1.0 mL). The mixture was heated at 140° C. for 5 minutes using microwave irradiation. The formed suspension was then diluted with EtOAc and washed with water and brine. Subsequent drying over Na₂SO₄ and evaporation of the solvent yielded a crude product that was purified over SiO₂ (EtOAc 90%, Et₃N 5%, MeOH 5%). A batch of 276 mg (78%) of the title compound was obtained. Mp 176.0-178.5° C.; ¹H-NMR (CDCl₃): δ (ppm) 7.46-7.34, (m, 3H), 5.30 (s, 2H), 3.86 (t, J=5.1 Hz, 4H), 2.44 (t, J=5.1 Hz, 4H), 2.31 (s, 3H).

### Example 2

### 2-(4-Methylpiperazinyl)-4-phenoxyquinazoline (VUF10327)

The following procedure is a representative procedure that may be followed for the synthesis of 2,4-disubstituted quinazolines.

2,4-Dichloroquinazoline (300 mg), 4-hydroxypyridine (145 mg) and NaH (69 mg) in DMF (5 mL) were stirred at room temperature. After 2 hours the reaction mixture was diluted with EtOAc and water. The aqueous phase was extracted with ethyl acetate and the combined organic layers were washed with H₂O and brine. After drying over Na₂SO₄ and evaporation of the solvent, the obtained residue was transferred to a microwave tube containing N-methylpiperazine (2 mL). The reaction mixture was heated at 140° C. for 5 minutes under microwave irradiation. The crude product was diluted with ethyl acetate and washed with H₂O and brine. After drying over Na₂SO₄ and subsequent evaporation of the solvent the residue was purified over SiO₂ (EtOAc 90%, Et₃N 5%, MeOH 5%) yielding 20 mg (4%) of product. Mp 102-103° C.; ¹H NMR (CDCl₃) δ 8.09 (d, J=8.2 Hz, 1H), 7.42 (m, 8H), 3.72 (t, J=4.9 Hz, 4H), 2.38 (t, J=5.0 Hz, 4H), 2.29 (s, 3H).

### Example 3

### 4-(Benzyloxy)-2-(4-methylpiperazinyl)-quinazoline (VUF10328)

The title compound was prepared according to the method of Example 2. Yield 163 mg (32%). ¹H NMR (CDCl₃) δ 7.94 (dd, J=0.9 Hz, J=8.1 Hz, 1H), 7.34 (m, BH), 5.52 (s, 2H), 3.93 (t, J=5.0 Hz, 4H), 2.48 (t, J=5.1 Hz, 4H), 2.33 (s, 3H).

### Example 4

### 2-(4-Methylpiperazinyl)-quinoline (VUF10049)

2-Chloroquinoline (328 mg) and N-methylpiperazine (1.5 mL) were added to a microwave tube and heated at 160° C. for 5 minutes. The obtained product was diluted with water and the aqueous layer was extracted with CHCl₃. The combined layers were dried over Na₂SO₄ and evaporated. Recrystallisation of the crude product from EtOAc/Hex yielded 274 mg (60%) of the title compound. Mp 110.3-112.4° C.; ¹H NMR (CDCl₃): δ (ppm) 7.89-7.85 (d, 1H, J=9.1 Hz), 7.70-7.66 (d, J=8.0, 1H), 7.59-7.47 (m, 2H), 7.20-7.17 (dd, J=1.2 Hz, J=6.9 Hz, 1H), 6.99-6.94 (d, J=9.1 Hz, 1H), 3.79-3.73 (t, J=5.1 Hz, 4H), 2.57-2.52 (t, J=5.1 Hz, 4H), 2.35 (s, 3H).

### Example 5

### 2-(4-Methylpiperazinyl)-quinoxaline (VUF10050)

2-Chloroquinoxaline (309 mg) and N-methylpiperazine (2.0 mL) were added to a microwave tube and heated at 160° C. for 5 minutes. The resulting mixture was diluted with water and extracted with EtOAc. Drying over Na₂SO₄ and evaporation of the solvent yielded 452 mg (98%) of product. Mp 108.6-110.9° C.; ¹H NMR (CDCl₃): δ (ppm) 8.56 (s, 1H), 7.85 (dd, J=1.3 Hz, J=8.2 Hz, 1H), 7.66 (dd, J=1.3 Hz, J=8.4 Hz, 1H), 7.59-7.50 (m, 1H), 7.41-7.24 (m, 1H), 3.79 (t, J=5.1 Hz, 4H), 2.54 (t, J=5.1 Hz, 4H), 2.34 (s, 3H).

### Example 6

### 6-Chloro-2-(4-methyl-piperazinyl)-quinoline (VUF6959)

2,6-Dichloroquinoline (300 mg) was added to N-methylpiperazine (2.0 mL) and was heated in the microwave at 160° C. for 5 min. After completion the obtained solution was evaporated to dryness, the residue was dissolved in EtOAc and then washed with saturated NaHCO₃ solution. The organic layer was dried with brine and Na₂SO₄. The product was recrystallised from EtOAc/Et₂O. Mp 141.1-142.5° C.; ¹H-NMR (DMSO): δ (ppm) 7.73 (d, J=9.2 Hz, 1H), 7.55 (d, J=8.9 Hz, 1H), 7.49 (d, J=2.3 Hz, 1H), 7.38 (dd, J=2.4 Hz, J=8.9 Hz, 1H), 6.92 (d, J=9.2 Hz, 1H), 3.69 (t, J=5.1 Hz, 4H), 2.48 (t, J=5.1 Hz, 4H), 2.29 (s, 3H).

### Example 7

### 6-Chloro-2-(4-methylpiperazinyl)-quinoxaline (VUF6886)

2,6-Dichloroquinoxaline (300 mg) and N-methylpiperazine (2.0 mL) were added to a microwave tube and heated at 160° C. for 5 minutes. After completion the obtained liquid was diluted with water and stirred for 20 minutes. The resulting suspension was filtered and washed with water. Residual water was removed by drying in vacuo, yielding 369 mg (94%) of product. Mp 151.2-151.8° C.; ¹H NMR (CDCl₃): δ (ppm) 8.55 (s, 1H), 7.84 (d, J=2.2 Hz, 1H), 7.59 (d, J=8.9 Hz, 1H), 7.48 (dd, J=2.2 Hz, J=8.9 Hz, 1H), 3.80 (t, J=5.0 Hz, 1H), 2.56 (t, j=5.1 Hz, 1H), 2.36 (s, 3H).

### Example 8

### 2-(4-Methylpiperazinyl)-quinazoline (VUF10249)

A solution of 2,4-dichloroquinazoline (1.2 g) in DCM (24 mL) covered with brine (24 mL) containing 9% NH₄OH was treated with powdered zinc (1.2 g) and the resulting biphasic mixture was then refluxed for 3.5 hours. After cooling to room temperature it was filtered through celite and the organic layer was removed under reduced pressure. The residue was diluted with EtOAc and washed with 1N HCl solution after which it was dried and concentrated to dryness. Without purification N-methylpiperazine (4.0 mL) was added. The product was diluted with ethyl acetate and washed with NaHCO₃ and water. Drying over Na₂SO₄ and rotary evaporation yielded the crude title compound. Purification over SiO₂ yielded 180 mg (13%) of product. Mp 74.4-76.0° C.; ¹H-NMR (CDCl₃): δ (ppm) 8.81 (s, 1H), 7.52-7.40 (m, 3H), 7.04-6.96 (m, 1H), 3.85 (t, J=5.0 Hz, 4H), 2.34 (t, J=5.0 Hz, 4H), 2.17 (s, 3H).

### Example 9

### 3-Trifluoromethylsulfonate-isoquinoline

Triflic anhydride (2.03 g) was added dropwise to a stirred solution of 3-hydroxy-isoquinoline (950 mg) in pyridine (7.0 mL) at 0° C. After 10 minutes the icebath was removed and the reaction was stirred at room temperature for 16 hours. The solution was then diluted with water and extracted with CHCl₃, the organic extracts were washed with brine and dried over Na₂SO₄. Evaporation of the solvents and purification over SiO₂ (EtOAc:Hex, 1:1) yielded 1.70 g (94%) of product. Mp 33.3-33.9° C. ¹H NMR (CDCl₃): δ (ppm) 9.04 (s, 1H), 8.03 (d, J=8.3 Hz, 1H), 7.90-7.62 (m, 3H) 7.55 (s, 1H).

### Example 10

### 3-(4-Methylpiperazinyl)-isoquinoline (VUF10047)

A microwave tube was charged with 500 mg of 3-trifluoromethylsulphonate-isoquinoline (500 mg) and N-methylpiperazine (1.5 mL). The tube was heated at 160° C. for 5 minutes and the obtained product was partitioned between CHCl₃ and water. Extraction of the aqueous layer and drying over Na₂SO₄, followed by evaporation of the solvent yielded the crude product. Purification over SiO₂ (EtOAc) yielded 264 mg (64%) of title compound. Mp 93.6-96.1° C.; ¹H NMR (CDCl₃): δ (ppm) 8.92 (s, 1H), 7.76 (d, J=8.1 Hz, 1H), 7.56 (d, J=8.1 Hz, 1H), 7.51-7.44 (m, 1H), 7.28-7.20 (m, 1H), 6.76 (s, 1H), 3.59 (t, J=5.1 Hz, 4H), 2.58 (t, J=5.1 Hz, 4H), 2.36 (s, 3H).

### Example 11

### 1-(4-Methylpiperazinyl)-isoquinoline (VUF10048)

1-Chloroisoquinoline (495 mg) and N-methylpiperazine (2.0 mL) were added to a microwave tube and heated at 160° C. for 5 minutes. The mixture was diluted with water and the aqueous layer was extracted with EtOAc. The combined organic layers were then dried with brine and Na₂SO₄. Evaporation of the solvent yielded 666 mg (97%) of product. ¹H NMR (CDCl₃): δ (ppm) 8.12 (d, J=5.8 Hz, 1H), 8.06 (d, J=8.4 Hz, 1H), 7.72 (d, J=7.5 Hz, 1H), 7.62-7.44 (m, 2H), 7.22 (d, J=5.8 Hz, 1H), 3.44 (t, J=4.8 Hz, 4H), 2.69 (t, J=4.8 Hz, 4H), 2.40 (s, 3H).

### Example 12

### 3-Benzyl-2-(4-methylpiperazinyl)-quinoxaline (VUF10148)

A microwave tube charged with 3-benzyl-2-chloroquinoxaline (400 mg) and N-methylpiperazine (3.0 mL) was heated at 120° C. for 5 minutes. Then water (25 mL) was added and the aqueous layer was extracted with EtOAc. The combined organic extracts were dried with brine and Na₂SO₄. Evaporation of the organic solvents and subsequent purification over SiO₂ (EtOAc 90%, Et₃N 5%, MeOH 5%) yielded 446 mg (89%) of product. Mp 89.0-90.0° C.; ¹H NMR (CDCl₃) δ (ppm) 7.82 (dd, J=1.4 Hz, J=8.3 Hz, 1H), 7.62-7.46 (m, 2H), 7.34-7.14 (m, 5H), 4.35 (s, 2H), 3.32 (t, J=4.8 Hz, 4H), 2.57 (t, J=4.8 Hz), 2.35 (s, 3H).

### Example 13

### 6,7-Dichloro-2-methoxy-3-(4-methylpiperazinyl)-quinoxaline (VUF10325)

2,3,6-Trichloro-3-(4-methylpiperazinyl)-quinoxaline (200 g) and a 5.4 M solution of NaOMe in MeOH (0.14 mL) were dissolved in MeOH (10 mL) and heated at reflux. After 2 hours the solution was diluted with CH₂Cl₂ and washed with water and brine. The organic layer was dried over Na₂SO₄ and evaporated to yield 199 mg (99%) of product. Mp 92.6-99.0° C.; ¹H NMR (CDCl₃): δ (ppm) 7.76 (s, 1H), 7.75 (s, 1H), 4.06 (s, 3H), 3.75 (t, J=5.0 Hz, 4H), 2.54 (t, J=5.1 Hz, 4H), 2.33 (s, 3H).

### Example 14

### 7-Chloro-2-methoxy-3-(4-methylpiperazinyl)-quinoxaline (VUF10321)

To a microwave tube were added 2,6-dichloro-3-methoxy-quinoxaline (200 mg), N-methylpiperazine (0.2 mL) and THF (3.0 mL). After heating the solution for 5 minutes at 140° C. the obtained suspension was diluted with EtOAc (30 mL) and washed with saturated NaHCO₃, water and brine. Drying with Na₂SO₄ and evaporation of the organic layer yielded the crude product. Purification over SiO₂ (EtOAc 90%, MeOH 5%, Et₃N 5%) yielded 168 mg (66%) of title compound. Mp 99.9-101.6° C.; ¹H-NMR (CDCl₃): δ (ppm) 7.66 (d, J=2.3 Hz, 1H), 7.59 (d, J=8.7 Hz, 1H), 7.34 (dd, J=8.8 Hz, J=2.4 Hz, 1H), 3.71 (t, J=5.0 Hz, 4H), 2.56 (t, J=5.0 Hz, 4H), 2.34 (s, 3H).

### Example 15

### 2,6-Dichloro-3-methoxy-quinoxaline

A solution of NaOMe in methanol (2.9 mL of a 5.4 M solution) was diluted with methanol (36 mL) and added over a 6 hour period to a slurry of 2,3,6-trichloroquinoxaline (3.0 g) in methanol (36 mL) at 50° C. After addition the obtained solution was refluxed for 16 hours and the solvent was evaporated. The residue was taken up in CHCl₃ (100 mL) and washed with water and brine. After drying over Na₂SO₄ the organic layer was removed and the residue was purified over SiO₂ (toluene) to yield crude product. After three crystallisations, 369 mg of the title compound was obtained. Mp 110.9-111.4° C.; ¹H-NMR (CDCl₃): δ (ppm) 7.86-7.82 (m, 2H), 7.51 (dd, J=8.8 Hz, J=2.2 Hz, 1H), 4.14 (s, 3H).

The following Example (Example 16) provides a general synthesis of 2,4-disubstituted quinazolines (General Method A).

### Example 16

### 6-Chloro-4-(N-methylamino)-2-(4-methylpiperazinyl)-quinazoline (VUF10349)

2,4,6-Trichloroquinazoline (300 mg) was dissolved in THF (5.0 ml) after which a solution of methylamine in water (0.12 ml, 40% w/w) was added to the solution. After 2 hours, the formed suspension was diluted with EtOAc (50 ml) and washed with water and brine. The organic phase was dried over Na₂SO₄ and concentrated (about 3 ml) after which it was transferred to a microwave tube containing N-methylpiperazine (1.0 ml) and EtOAc (3.0 ml). The mixture was heated at 140° C. for 5 minutes with microwave irradiation. The product was then diluted with EtOAc and washed with water and brine. Subsequent drying over Na₂SO₄ and evaporation of the solvent yielded an oil that was purified over SiO₂ (EtOAc 90%, Et₃N 5%, MeOH 5%). The title compound was obtained as a solid. Yield: 308 mg (82%). Mp 173.2-175.7° C.; ¹H-NMR (CDCl₃): δ (ppm) 7.42-7.24 (m, 3H), 5.42 (s, 1H), 3.92 (t, J=4.8 Hz, 4H), 3.10 (d, J=4.8 Hz, 3H), 2.46 (t, J=4.8 Hz, 4H), 2.32 (d, J=1.0 Hz, 3H); ¹³C NMR (CDCl₃) δ (ppm) 159.47, 158.84, 150.49, 132.73, 127.27, 125.42, 119.99, 110.98, 55.03, 46.12, 43.61, 27.96; MS (ESI) m/z 292 (M+H)⁺.

### Example 17

### 6-Chloro-2-(4-methylpiperazinyl)-4-phenylethylamino-quinazoline (VUF10453)

General method A. Yield 212 mg (65%). Mp 136.6-138.8° C.; ¹H NMR (CDCl₃) δ 7.38-7.19 (m, 8H), 5.42 (app br t, 1H), 3.94-3.89 (m, 4H), 3.80 (q, J=6.9 Hz, 2H), 2.97 (t, J=7.1 Hz, 2H), 2.46 (t, J=5.1 Hz, 4H), 2.32 (s, 3H); ¹³C NMR (CDCl₃) δ (ppm) 158.80, 159.70, 150.70, 138.81, 132.80, 128.62, 128.60, 127.34, 126.48, 125.45, 119.86, 110.83, 54.99, 46.10, 43.62, 42.30, 35.10; MS (ESI) m/z 382 (M+H)⁺.

### Example 18

### 6-Chloro-4-isopropylamino-2-(4-methylpiperazinyl)-quinazoline (VUF10452)

General method A. Yield: 227 mg (83%). Mp 187.8-189.8° C.; ¹H-NMR (CDCl₃) δ (ppm) 7.42-7.31 (m, 3H), 5.13 (d, J=6.6 Hz, 1H), 4.39 (sp, J=6.6 Hz, 1H), 3.88 (t, J=4.9 Hz, 4H), 2.45 (t, J=5.0 Hz, 4H), 2.31 (s, 3H), 1.29 (d, J=6.5 Hz, 6H); ¹³C NMR (CDCl₃) δ 158.88, 157.98, 150.69, 132.63, 127.34, 125.31, 119.89, 110.82, 55.00, 46.12, 43.59, 42.68, 22.35; MS (ESI) m/z 320 (M+H)⁺.

### Example 19

### 6-chloro-2-(4-methylpiperazinyl)-4-(2-hydroxyethylamino)-quinazoline (VUF10455)

General method A. The compound was purified by recrystallisation from EtOAc. Yield: 207 mg (75%) of a solid. Mp 188.7-190.3° C.; ¹H NMR (DMSO-δ₆) δ (ppm) 8.14 (d, J=2.3 Hz, 1H), 7.48 (dd, J=2.2 Hz, J=8.9 Hz, 1H), 7.25 (d, J=8.9 Hz, 1H), 4.76 (t, J=5.4 Hz, 1H), 3.75 (app br s, 4H), 3.63-3.51 (m, 4H), 2.33 (t, J=4.6 Hz, 4H), 2.19 (s, 3H); ¹³C NMR (DMSO-δ₆) δ (ppm) 159.94, 158.40, 132.18, 126.67, 123.67, 121.89, 111.09, 58.68, 54.36, 45.36, 43.15, 43.04; MS (ESI) m/z 322 (M+H)⁺.

### Example 20

### Ethyl 2-(6-chloro-2-(4-methylpiperazin-1-yl)quinazolin-4-ylamino)acetate (VUF10456)

General method A. The compound was purified by recrystallisation from EtOAc. Yield: 150 mg (42%) of a solid. Mp 108.5-109.7° C.; ¹H-NMR (DMSO-δ₆) δ (ppm) 8.65 (m, 1H), 8.12 (d, J=2.3 Hz, 1H), 7.52 (dd, J=2.3 Hz, J=9.0 Hz, 1H), 7.27 (d, J=9.0 Hz, 1H), 4.14-4.03 (m, 4H), 3.69 (m, 4H), 3.33-3.31 (m, 2H), 2.29 (m, 4H), 2.18 (s, 3H), 1.16 (t, J=7.1 Hz, 3H); ¹³C NMR (DMSO-δ₆) δ 169.86, 158.83, 157.94, 150.26, 132.60, 126.80, 123.97, 121.86, 110.73, 60.04, 54.29, 45.62, 42.91, 13.91; MS (ESI) m/z 364 (M+H)⁺.

### Example 21

### 7-Chloro-4-methylamino-2-(4-methylpiperazinyl)-quinazoline (VUF10458)

General method A. As a precursor for this reaction, 2,4,7-trichloroquinazoline was used. Yield: 219 mg (83%). Mp 177.8-179.8° C.; ¹H-NMR (CDCl₃) δ (ppm) 7.39 (d, J=2.0 Hz, 1H), 7.30 (d, J=8.6 Hz, 1H), 6.93 (dd, J=2.1 Hz, J=8.6 Hz, 1H), 3.91 (t, J=5.0 Hz, 4H), 3.08 (d, J=4.8 Hz, 3H), 2.44 (t, J=5.1 Hz, 4H), 2.30 (s, 3H); ¹³C NMR (CDCl₃) δ 159.98, 159.22, 153.02, 138.10, 124.81, 121.76, 121.08, 108.78, 55.03, 46.14, 43.58, 27.90.

### Example 22

### 6-Chloro-2-(4-methylpiperazin-1-yl)-N-(4-nitrophenethyl)quinazolin-4-amine (VUF10506)

General method A. MS (ESI) m/z 427 (M+H)⁺.

### Example 23

### N-(4-aminobenzyl)-6-chloro-2-(4-methylpiperazin-1-yl)quinazolin-4-amine (VUF10509)

General Method A. Mp 145.9-148.5° C.; ¹H-NMR (CDCl₃) δ (ppm) 7.37-7.32 (m, 3H), 7.15 (d, J=8.4 Hz, 2H), 6.65 (d, J=8.4 Hz, 2H), 5.47 (m, 1H), 4.60 (d, J=5.0 Hz, 2H), 3.91 (t, J=5.0 Hz, 4H), 2.45 (t, J=5.0 Hz, 4H), 2.31 (s, 3H); ¹³C NMR (CDCl₃) δ 158.53, 158.40, 150.34, 147.34, 132.21, 128.40, 126.68, 126.11, 123.71, 121.87, 113.36, 111.11, 54.38, 45.67, 43.36, 43.09; MS (ESI) m/z 383 (M+H)⁺.

### Example 24

### 6-Chloro-N-(4-methoxybenzyl)-2-(4-methylpiperazin-1-yl)quinazolin-4-amine (VUF10505)

General method A. ¹H-NMR (CDCl₃) δ (ppm) 7.35-7.19 (m, 5H), 6.82 (d, J=7.7 Hz, 2H), 5.61 (m, 1H), 4.62 (d, J=5.0 Hz, 2H), 3.86 (t, J=4.5 Hz, 4H), 3.74 (s, 3H), 2.41 (t, J=4.6 Hz, 4H), 2.27 (s, 3H); ¹³C NMR (CDCl₃) δ 158.95, 158.73, 158.55, 150.67, 132.83, 130.20, 129.16, 127.25, 125.49, 120.02, 113.95, 110.74, 55.14, 54.92, 46.04, 44.64, 43.58; MS (ESI) m/z 398 (M+H)⁺.

### Example 25

### 6-Chloro-N-(4-fluorobenzyl)-2-(4-methylpiperazin-1-yl)quinazolin-4-amine (VUF10513)

General method A. Mp 127.4-131.5° C.; ¹H-NMR (CDCl₃) δ (ppm); 7.40-7.19 (m, 5H), 7.01-6.92 (m, 2H), 5.68 (m, 1H), 4.67 (d, J=5.4 Hz, 2H), 3.84 (t, J=4.8 Hz, 4H), 2.39 (t, J=5.1 Hz, 4H), 2.27 (s, 3H); MS (ESI) m/z 386 (M+H)⁺.

### Example 26

### 4-((6-Chloro-2-(4-methylpiperazin-1-yl)quinazolin-4-ylamino)methyl)benzonitrile (VUF10553)

General method A. Mp 127.4-131.5° C.; ¹H-NMR (CDCl₃) δ (ppm) 7.60 (d, d=8.4 Hz, 2H), 7.47-7.35 (m, 5H), 5.85 (m, 1H), 4.82 (d, J=5.7 Hz, 2H), 3.80 (t, J=5.0 Hz, 4H), 2.38 (t, J=5.0 Hz, 4H), 2.29 (s, 3H); ¹³C NMR (CDCl₃) δ 158.63, 158.16, 150.36, 145.53, 132.50, 131.97, 127.87, 126.80, 123.94, 121.82, 118.70, 110.88, 109.13, 54.22, 45.60, 43.53, 43.04; MS (ESI) m/z 393 (M+H)⁺.

### Example 27

### 6-Chloro-N-(3,4-difluorobenzyl)-2-(4-methylpiperazin-1-yl)quinazolin-4-amine (VUF10556)

General method A. Mp 127.4-131.5° C.; ¹H-NMR (CDCl₃) δ (ppm); 7.49 (d, J=1.6 Hz, 1H), 7.44-7.33 (m, 2H), 7.19-7.03 (m, 3H), 5.97 (m, 1H), 4.68 (d, J=5.6 Hz, 2H), 3.85 (t, J=5.0 Hz, 4H), 2.43 (t, J=5.0 Hz, 4H), 2.30 (s, 3H); ¹³C NMR (CDCl₃) δ 158.59, 158.47, 150.71, 135.46, 133.12, 127.36, 125.78, 123.51, 123.43, 123.38, 123.31, 119.99, 117.41, 117.07, 116.69, 116.35, 110.57, 54.82, 45.98, 44.09, 44.06, 43.55.

### Example 28

### 2,6-Dichloro-N-methylquinazolin-4-amine

To a suspension of 2,4,6-trichloroquinazoline (4.7 g) in EtOH (150 ml) was added methylamine (1.91 ml of 40% w/w in water). The mixture was then stirred at r.t. for 40 minutes after which the solution was concentrated to a volume of about 30 ml. The reaction mixture was then diluted with water (200 ml) and extracted with EtOAc. The combined organic layers were washed with brine and dried over Na₂SO₄. Evaporation of the solvent yielded 3.90 g (85%)g of a solid that was used in the next step without further purification.

The following Example (Example 29) provides a general synthesis of 2,4-diamino-substituted quinazolines (General Method B).

### Example 29

### 6-Chloro-N-methyl-2-(4-methyl-1,4-diazepan-1-yl)quinazolin-4-amine (VUF10434)

A microwave tube was charged with 2,6-dichloro-4-methylaminoquinazoline (100 mg), diisopropylethylamine (113 μl), N-methylpyrrolidinone (200 μl) and homopiperazine (75 mg, or 1.5 equivalents of another amine). The mixture was then heated at 150° C. for 10 minutes and the obtained solution was transferred to a LC-MS vial using a small amount of N-methylpyrrolidinone. The reaction mixture was directly purified with preparative LC-MS and freeze dried to yield the desired title compound.

Boc-protected intermediates (for VUF10438 and VUF10440) were deprotected in 4 M of HCl in dioxane until LCMS indicated complete conversion to the final unprotected product. All final compounds were analyzed with LCMS under condition II.

The following compounds in Table 5 were synthesised from 2,6-dichloro-4-methylamino-quinazoline according to General Method B.

TABLE 5

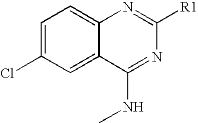

| | | | | Rₜ | (M + |
|---|---|---|---|---|---|
| Nr | Code | R₁ | MW | (min)^{a} | H⁺)^{b} |
| 30 | VUF10427 | 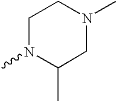 | 305 | 1.78 | 306 |
| 31 | VUF 10428 | 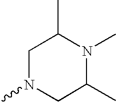 | 319 | 1.78 | 320 |
| 32 | VUF 10429 | 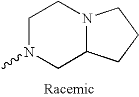 | 317 | 1.79 | 318 |
| 33 | VUF 10432 | 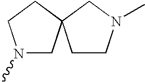 | 331 | 1.71 | 332 |
| 34 | VUF 10433 | 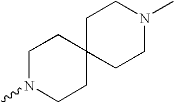 | 359 | 1.82 | 360 |
| 35 | VUF 10434 | 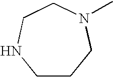 | 305 | 1.71 | 306 |
| 36 | VUF 10438 | 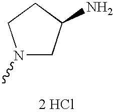 | 363 | 1.51 | 364 |
| 37 | VUF 10440 | 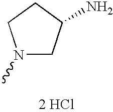 | 363 | 1.51 | 364 |

^{a}The LCMS conditions can be found above (condition II).
^{b}(M + H⁺) is the found compound mass by MS.

### Example 38

Radioligand displacement studies at the human H₄ receptor were conducted. More particularly, HEK 293T cells were maintained in Dulbecco's modified Eagle medium (DMEM) supplemented with 10% fetal bovine serum (FBS), 50 IU/ml penicillin, and 50 μg/ml streptomycin in 5% CO2 humidified atmosphere at 37° C. Approximately 4 million cells were seeded in a 10-cm dish and cultured overnight before transfection. For transfection of each dish of cells, the transfection mixture was prepared in 1 ml serum-free DMEM and contained 5 μg of human H₄R receptor plasmid and 15 μl of 1 mg/ml 25 kDa linear polyethyleneimine (Polyscience, Inc., USA). The mixture was incubated for 10-15 minutes at room temperature before it was added into the monoloyer cell culture loaded with 5 ml fresh cell culture medium. Two days after transfection the cells were washed with PBS containing 1 mM EDTA, collected as pellet by centrifuging, and stored at −20° C. until use.

For the radioligand binding study, pellets of transfected cells were homogenized in H₄R binding buffer (100 mM Tris-HCl, pH 7.4). For displacement binding assays, the membranes were typically incubated with 10⁻⁴ to 10⁻¹¹ M of ligands (stock concentration was 10 mM 1 DMSO) in the presence of [³H]histamine (Perkin-Elmer Life Science, Inc., USA) in a total volume of 200 μl. The reaction mixtures were incubated for 1 hour at room temperature (22° C.), and harvested on 96-well glass fiber C plates that were pretreated with 0.3% 750 kDa PEI. The binding assay data were analyzed using Prism 4.0 (Graphpad Software Inc., USA).

Compounds of the invention, including VUF10249, 10319, 10321, 10323, 10325, 10329, 10330, 10334, 10348, 10349, 10350, 10351, 10352, 10353, 10170, 10199, 10277, 6686, 10144, 10059, 10180, 10274, 10453, 10452, 10455, 10456, 10506, 10509, 10505, 10513, 10553, 10556 and 6959, demonstrated a Kᵢ value of <1 μM. Other compounds demonstrated somewhat higher values.

### Example 39

The affinity of compounds of the present invention was tested on CHO (Chinese Hamster Ovary) cells. Binding analysis was performed using crude cell homogenates of CHO cells, stably transfected with a synthetic gene encoding the human histamine H₄ receptor (GENEart Gmbh, Regensburg, Germany), in 50 mM Tris-HCl buffer pH 7.4 and approximately 7 nM [³H]histamine (18.1 Ci/mmol, Perkin-Elmer), with or without competing ligands in a total volume of 200 μL as essentially described by Lim et al., J. Pharmacol. Exp. Ther. 2005, 314, 1310-1321. Bound radioligand was collected on 0.3% polyethyleneimine-pretreated 96-well GF/C plates, and washed three times with 0.3 mL of ice-cold washing buffer containing 50 mM Tris-HCl (pH 7.4 at 4° C.).

### Example 40

The functional profile of compounds of the present invention was tested on HEK (Human Embryonic Kidney) cells. HEK 293T cells were transiently transfected with H₄ receptor cDNA and a CRE-β-galactosidase reporter plasmid (pCRE/β-gal, Chen et al., 1995) using polyethyleneimine. After 48 hours, the cells were exposed for six hours to putative H₄ receptor ligands in the absence or presence of histamine in serum-free DMEM (Dulbecco's Modified Eagle's Medium) medium containing 1 μM forskolin. Thereafter the beta-galactosidase activity was determined as essentially described by Lim et al., J. Pharmacol. Exp. Ther. 2005, 314, 1310-1321, and known to those skilled in the art.

1. A compound of formula (I) 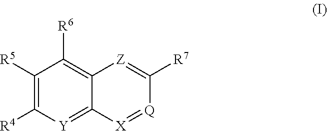
wherein
Q is CR¹ or N;
X is CR² or N, provided that Q and X are not both N;
Y is CR³ or N;
Z is CH or N;
R¹, R², R³, R⁴, R⁵ and R⁶ are independently H, F, Cl, Br, I, or a hydrocarbon group which optionally contains one or more heteroatoms; and
R⁷ is a heterocyclic radical including one or more N atoms; or a pharmaceutically acceptable salt, ester or solvate thereof.

2. The compound according to claim 1, wherein R¹ and R² are independently selected from H, F, Cl, Br, I, C₁₋₄ alkyl, C₂₋₅ alkenyl, C₁₋₄ alkoxy, cycloalkyl, aryl, heteroaryl, —C₁₋₄ alkyl-aryl, C₁₋₄ alkyl-heteroaryl, arylethyl O-aryl, O-heteroaryl, NH-aryl, NH-heteroaryl, S-aryl, S-phenyl, S-heteroaryl, O—C₁₋₄ alkyl-aryl, O—C₁₋₄ alkyl-heteroaryl, O—C₁₋₄ alkyl-aryl, NH—C₁₋₄ alkyl-aryl, NH—C₁₋₄ alkyl-heteroaryl, hydrazino hydroxylamino, NH₂, O—C₁₋₄ alkyl-N(CH₃)₂ or NR^{a}R^{b},
wherein either R^{a} is absent and R^{b} is acyl, or each of R^{a} and R^{b} is independently selected from H, C₁₋₄ alkyl, cycloalkyl, phenyl, benzyl or phenethyl, and wherein any of said aryl, heteroaryl, alkyl, acyl, phenyl and cycloalkyl moieties is optionally substituted with 1 to 3 substituents selected from C₁₋₄ alkyl, halogen, hydroxyl, amino and C₁₋₃ alkoxy.

3. The compound according to claim 1, wherein R³, R⁴, R⁵ and R⁶ are independently selected from H, F, Cl, Br, I, C₁₋₄ alkyl, C₂₋₅ alkenyl, C₂₋₅ alkynyl, C₁₋₄ alkoxy, C₁₋₄ alkylthio, C₃₋₆ cycloalkyl, O—C₃₋₆ cycloalkyl, phenyl, benzyl, O-phenyl, NH-phenyl, S-phenyl, O—C₁₋₄ alkyl-phenyl, C₁₋₄ alkyl-phenyl, CF₃, O—CF₃, S—CF₃, hydroxy, nitro, cyano, O—C₁₋₄ alkyl-N(CH₃)₂, and NR^{a}R^{b}, wherein R^{a} and R^{b} are independently selected from H, C₁₋₄ alkyl, phenyl, benzyl and phenethyl, and wherein any phenyl, alkyl or cycloalkyl moiety is optionally substituted with 1 to 3 substituents selected from C₁₋₃ alkyl, halogen, hydroxy, amino and C₁₋₃ alkoxy.

4. The compound according to claim 3, wherein at least three of R³, R⁴, R⁵ and R⁶ are H.

5. The compound according to claim 1, wherein R⁷ is selected from 4-7 membered heterocyclyl, C₃₋₇ cycloalkyl-4-7 membered heterocyclyl and bis-(4-7 membered heterocyclyl).

6. The compound according to claim 1, wherein Q is CR¹, X is N, Y is CR³, and Z is N.

7. The compound according to claim 6, wherein R³, R⁴, R⁵ and R⁶ are each H.

8. The compound according to claim 6, wherein R⁷ is 4-methylpiperazino.

9. The compound according to claim 6, wherein R⁷ is 4-methyl-1,4-diazepane, 3-methylamino-azetidine or (3-methylamino)pyrrolidone.

10. The compound according to claim 1, wherein Q is N, X is CR², Y is CR³ or N, and Z is N.

11. The compound according to claim 10, wherein R⁷ is 4-methylpiperazino.

12. The compound according to claim 1, wherein Q, X and Y are CH, and Z is N.

13. The compound according to claim 12, wherein R⁵ and R⁶ are each H.

14. The compound according to claim 1, wherein X, Y and Z are CH, and Q is N.

15. The compound according to claim 14, wherein R⁴, R⁵ and R⁶ are each H.

16. The compound according to claim 1, selected from 4-amino-6-chloro-2-(4-methylpiperazinyl)-quinazoline, 2-(4-methylpiperazinyl)-4-phenoxyquinazoline, 4-(benzyloxy)-2-(4-methylpiperazinyl)-quinazoline, 2-(4-methylpiperazinyl)-quinoline, 2-(4-methylpiperazinyl)-quinoxaline, 6-chloro-2-(4-methyl-piperazinyl)-quinoline, 6-chloro-2-(4-methylpiperazinyl)-quinoxaline, 2-(4-methylpiperazinyl)-quinazoline, 3-(4-methylpiperazinyl)-isoquinoline, 1-(4-methylpiperazinyl)-isoquinoline, 3-benzyl-2-(4-methylpiperazinyl)-quinoxaline, 6,7-dichloro-2-methoxy-3-(4-methylpiperazinyl)-quinoxaline and 7-chloro-2-methoxy-3-(4-methylpiperazinyl)-quinoxaline.

17. A method for providing therapy for a condition which involves antagonism, inverse agonism or partial agonism at the histamine H₄ receptor wherein said method comprises administering, to a subject in need of such therapy, a compound of claim 1.

18. A method for providing therapy for a disorder or discomfort in a subject which is a response to a physical stimulus, a chemical stimulus, infection, an invasion by a body that is foreign to said subject, allergy, asthma, chronic obstructed pulmonary disease, atherosclerosis, rheumatoid arthritis, multiple sclerosis, inflammatory bowel disease, myasthenia gravis, autoimmune neuropathy, autoimmune uveitis, autoimmune haemolytic anemia, pernicious anemia, autoimmune thrombocytopenia, temporal arteritis, anti-phospholipid syndrome, vasculitides, Behcet's disease, dermatitis herpetiformis, pemphigus vulgaris, vitiligio, primary biliary cirrhosis, autoimmune hepatitis, autoimmune oophoritis, autoimmune orchitis, autoimmune disease of the adrenal gland, polymyositis, dermatomyositis, spondyloarthropathy, or Sjogren's syndrome, wherein said method comprises administering, to a subject in need of such therapy, a compound of claim 1.

19. A method for providing therapy for acute inflammation, allergic inflammation, chronic inflammation, Crohn's disease, ulcerative colitis, psoriasis, allergic rhinitis, scleroderma, autoimmune thyroid disease, immune-mediated diabetes mellitus, cancer, itch or lupus, wherein said method comprises administering, to a subject in need of such therapy, a compound of claim 1.

20. A pharmaceutical composition comprising a compound according to claim 1 and at least one pharmaceutically acceptable additive.

21. (canceled)
